(19) United States
(12) Patent Application Publication
(10) Pub. No.: US 2014/0132929 A1
(43) Pub. Date: May 15, 2014

(54) OPHTHALMOLOGIC APPARATUS, OPHTHALMOLOGIC EXAMINATION METHOD, AND PROGRAM

(71) Applicant: CANON KABUSHIKI KAISHA, Tokyo (JP)

(72) Inventor: Toshiya Fujimori, Kawasaki-shi (JP)

(73) Assignee: CANON KABUSHIKI KAISHA, Tokyo (JP)

(21) Appl. No.: 14/066,955

(22) Filed: Oct. 30, 2013

(30) Foreign Application Priority Data
Nov. 9, 2012 (JP) 2012-247026

(51) Int. Cl. A61B 3/12

(52) U.S. Cl. CPC A61B 3/12
USPC 351/211; 351/246

(57) ABSTRACT

Provided is an ophthalmologic examination method including a first control step of driving a focus unit based on an index image acquired by projecting an index to an eye to be inspected in a state of setting light intensity for illuminating the eye to be inspected to a first light intensity, a second control step of driving the focus unit based on the index image acquired in a state of setting the light intensity to a second light intensity higher than the first light intensity, and a memory step of storing specific information on the eye to be inspected during the first control step and the second control step. The second control step is executed after driving the focus unit to a position stored in the memory step in the case of storing again the specific information after storing the specific information.

### BACKGROUND OF THE INVENTION

1. Field of the Invention

The present invention relates to an ophthalmologic apparatus, an ophthalmologic examination method, and a program.

2. Description of the Related Art

In fundus photographing using a fundus camera, an alignment adjustment to appropriately match a positional relation between the fundus camera and an eye to be inspected and a focus adjustment to a fundus are executed before photographing so as to photograph an excellent fundus image.

In a mode for executing the observation of an anterior ocular segment of the eye to be inspected and a mode for executing at least one of the observation and the photographing of the fundus of the eye to be inspected, there is proposed a method for focus adjustment that involves controlling a focus lens to be at a predetermined position in accordance with a switched mode (see Japanese Patent Application Laid-Open No. 2012-50582).

Further, there is proposed an automatic focus function for automatically executing the focus adjustment, which has been manually executed by an examiner, using a focus index projected to the fundus of an eye to be inspected (see Japanese Patent Application Laid-Open No. 2009-172157).

The automatic focus function using a related art focus index has executed automatic focus processing without using specific information on the eye to be inspected, for example, a focus position of the eye to be inspected based on a history of fundus examination information. Thus, there has been a problem that the automatic focus processing takes time.

Further, a fundus observation image is captured without dimming illumination light which illuminates the fundus of the eye to be inspected so as to execute an automatic focus control using the image. This leads to a possibility that harmful light such as object lens reflection of illumination light and scattered reflection from the fundus of the eye to be inspected is simultaneously captured, thereby causing a detection error. However, the dimming of the illumination light makes the detection error small, and even when the focus index is blurred and low in luminance in the case where the dimmed harmful light is away from the focus position, a detection possibility is increased, and a trade-off of lowering accuracy of a photometric value (AE, AutoExposure) used to determine a photographing light intensity using the reflection light of the fundus occurs.

### SUMMARY OF THE INVENTION

The present invention has been made in view of the above-mentioned problems, and the present invention is to provide an ophthalmologic apparatus, an ophthalmologic examination method, and a program capable of compatibly speeding up automatic focus processing and maintaining an AE (AutoExposure) accuracy when executing the automatic focus for the eye to be inspected of one and the same person.

An ophthalmologic apparatus according to one embodiment of the present invention includes:

a memory unit for storing specific information on an eye to be inspected;

an illumination unit for illuminating the eye to be inspected;

a projection unit for projecting an index to the eye to be inspected;

an index image acquire unit for acquiring an index image based on return light from the eye to be inspected;

a drive unit for driving a focus unit based on the index image;

a light, intensity control unit for controlling light intensity that illuminates the eye to be inspected from the illumination unit;

a focus control unit for executing a first control for controlling the light intensity control unit to change the light intensity into a first light intensity and controlling the drive unit to drive the focus unit based on the index image acquired, in a state of changing the light intensity into the first light intensity and a second control for controlling the light intensity control unit to change the light intensity into a second light intensity higher than the first light intensity and controlling the drive unit to drive the focus unit based on the index image acquired in a state of changing the light intensity into the second light intensity; and

a memory control unit for controlling the memory unit to store the specific information during the first control and the second control,

in which the focus control unit executes the second control after the focus unit is driven to a position stored in the memory unit in a case where the memory control unit controls the memory unit to store the specific information and thereafter controls the memory unit to store the specific information again.

Further, an ophthalmologic examination method according to one embodiment, of the present invention includes:

a first control step of changing light intensity for illuminating an eye to be inspected into a first light intensity and driving a focus unit based on an index image acquired by projecting an index to the eye to be inspected in a state of changing the light intensity into the first light intensity;

a second control step of changing the light intensity into a second light intensity higher than the first light intensity and driving the focus unit based on the index image acquired in a state of changing the light intensity into the second light intensity; and

a memory step of storing specific information on the eye to be inspected during the first control step and the second control step,

in which the second control step is executed after driving the focus unit to a position stored in the memory step in a case where the specific information is stored again after the specific information is stored in the storage step.

According to the present invention, compatibility between speeding up of the automatic focus processing and maintaining the AE (AutoExposure) accuracy can be established by executing the focus control using the specific information on the eye to be inspected.

Further features of the present invention will become apparent from the following description of exemplary embodiments with reference to the attached drawings.

### BRIEF DESCRIPTION OF THE DRAWINGS

FIG. 1 is a configuration diagram of a fundus camera according to one embodiment of the present invention.

FIGS. 2A, 2B and 2C are diagrams illustrating the operation at the time of acquiring specific information on an eye to be inspected of the fundus camera according to one embodiment of the present invention.

FIG. 3 is a diagram illustrating the fundus examination operation in the case where specific information on the eye to be inspected is stored and not stored in a memory unit of the fundus camera according to one embodiment of the present invention.

FIG. 4 is a diagram illustrating a photographing flow at the time of multiple photographing of the same eye of the same subject of the fundus camera according to one embodiment of the present invention.

FIG. 5 is a diagram illustrating a photographing flow at the time of switching to a left eye or a right eye after photographing the fundus of the same subject of the fundus camera according to one embodiment of the present invention.

FIG. 6 is a diagram illustrating the operation at the time of searching for the specific information on an eye to be inspected by a search unit of the fundus camera according to one embodiment of the present invention.

FIGS. 7A, 7B and 7C are schematic diagrams illustrating a relation between the appearance of a focus index image displayed on a monitor of the fundus camera according to one embodiment of the present invention and a state in which focus index light beams reach the fundus Er of the eye to be inspected.

### DESCRIPTION OF THE EMBODIMENTS

Preferred embodiments of the present invention will now be described in detail in accordance with the accompanying drawings.

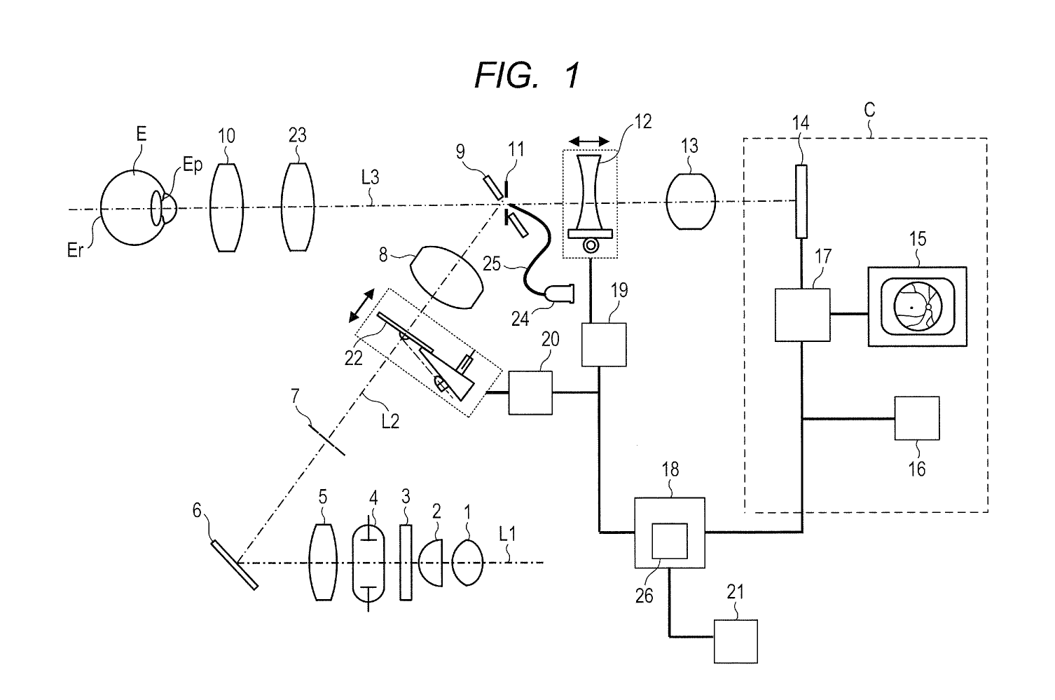

A configuration of a fundus camera, which is one embodiment of the present invention and an example of an ophthalmologic apparatus, is described with reference to FIG. 1.

### First Embodiment

FIG. 1 illustrates a configuration example of a fundus camera which is an ophthalmologic apparatus embodying the present invention.

There are arranged on an optical axis L1 an observation light source 1 for emitting stationary light such as a halogen lamp, a condenser lens 2, a filter 3 transmitting infrared light and blocking visible light, a photographing light source A such as an electronic flash, a lens 5, and a mirror 6. Further, there are sequentially arranged on an optical axis L2 in the direction of reflection of the mirror 6 a ring diaphragm 7 having a ring-shaped opening, a relay lens 8, and a perforated mirror 9 having a central portion opening.

Further, there are arranged on an optical axis L3 in the direction of reflection of the perforated mirror 9 an anterior ocular segment observations lens 23 and an objective lens 10 placed to face an eye E to be inspected. In a hole of the perforated mirror 9, a photographing diaphragm 11 is arranged. Further, behind the photographing diaphragm 11, there are sequentially arranged a focus lens 12 for adjusting focus by moving its position on the optical axis L3 and a photographing lens 13. Further, a working dot (WD) light source 24 for projecting an alignment index to the cornea Ep of the eye to be inspected is connected to the perforated mirror 9 via a fiber 25.

Ahead of the photographing lens 13, there is sequentially arranged an image pickup element 14 not only for observing moving images but also for imaging still images within a photographing camera C. An output of the image pickup element 14 is connected to an image processing portion 17, and an output of the image processing portion 17 is connected to a system control portion 18. The image processing portion 17 projects an observation image photographed by the image pickup element 14 to a monitor 15. Further, the system control portion 18 includes a memory unit 26 for storing the specific information on the eye to be inspected. The image processing portion 17 and the system control portion 18 are connected to a photographing control portion 16, and the photographing operation in the image processing portion 17 is executed in response to the instruction from the photographing control portion 16.

Meanwhile, a focus index projection portion 22 is arranged between, the ring diaphragm 7 and the relay lens 8 on the optical axis L2. Note that, the focus index projection portion 22 and the focus lens 12 are respectively moved simultaneously in the direction, of the optical axis L2 and the optical axis L3 by a focus lens drive portion 19 and a focus index drive portion 20 based on the control from, the system control portion 18. This system control portion 18 controls the focus lens drive portion 19 and the focus index drive portion 20 in accordance with the operation input of an operation input portion 21 in a manual focusing mode. At this time, the focus index projection portion 22 and the image pickup element 14 nave optically a conjugate relation. In addition, the system control portion 18 performs control of the light intensity adjustment and turning on/off of the observation light source 1 and also control of the light intensity adjustment and turning on/off of the photographing light source 4.

Further, the fundus camera according to this embodiment includes a fundus observation and photographing mode and an anterior ocular segment observation mode. In addition, the fundus camera includes color photographing and FAF photographing using autofluorescence of the fundus as a type of photographing.

Note that, the above-mentioned observation light source 1 constructs an illumination unit for illuminating the eye to be inspected in the present invention, the focus index projection portion 22 constructs a projection unit for projecting an index to the eye to be inspected, and the image pickup element 14 constructs an index image acquire unit for acquiring an index image based on return light from the eye to be inspected. Further, the focus lens drive portion 19 constructs a drive unit for driving the focus lens 12 which is a focus unit based on the index image, and the system control portion 18 for executing control such as a light intensity adjustment of the observation light source 1 and the photographing light source 4 constructs a light intensity control unit for controlling the illumination intensity of the eye to be inspected by the illumination unit.

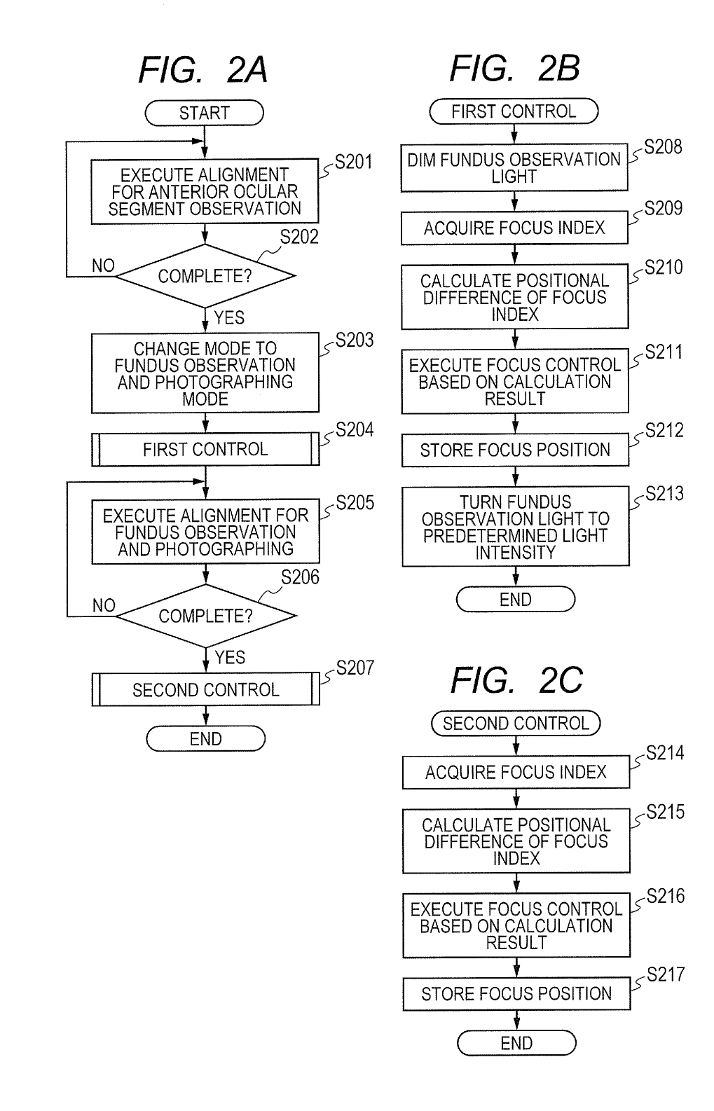

Next, the operation at the time of acquiring the specific information on the eye to be inspected is described with reference to FIGS. 2A to 2C.

FIGS. 2A to 2C are flowcharts illustrating the operation at the time of acquiring the specific information on the eye to be inspected of the fundus camera according to one embodiment of the present invention. FIG. 2A illustrates a main flow, FIG. 2B illustrates a first control flow, and FIG. 2C illustrates a second control flow.

An examiner executes the alignment between the anterior ocular segment of the eye E to be inspected and the fundus camera with respect to a horizontal direction (X direction), a vertical direction (Y direction), and a front-back direction (Z direction), while observing the anterior ocular segment of the eye E to be inspected displayed on the monitor 15 (S201). When the alignment between the anterior ocular segment of the eye E to be inspected and the fundus camera is completed (S202), the examiner executes the operation of retracting the anterior ocular segment observation lens 23 from above the optical axis L3 (S203). These operations control the examiner to observe a fundus Er of the eye to be inspected. At this juncture, the fundus camera is switched from the anterior ocular segment observation mode to the fundus observation and photographing mode.

In a first control (S204), the system control portion 18 dims the observation light source 1 (S208). The focus index image of the focus index projection portion 22 photographed by the image pickup element 14 is acquired in a state of being dimmed (S209), and the system control portion 18 calculates the positional difference of the focus index image (S210). Based on the calculation result, the system control portion 18 controls the focus lens drive portion 19 and the focus index drive portion 20 (S211). After controlling both drive portions 19 and 20, the system control portion 16 stores the current focus position (S212), and turns the observation light source 1 to a predetermined light intensity (S213), thereby completing the first control.

Here, the processing of S210, namely the calculation of the positional difference of the focus index image, is described.

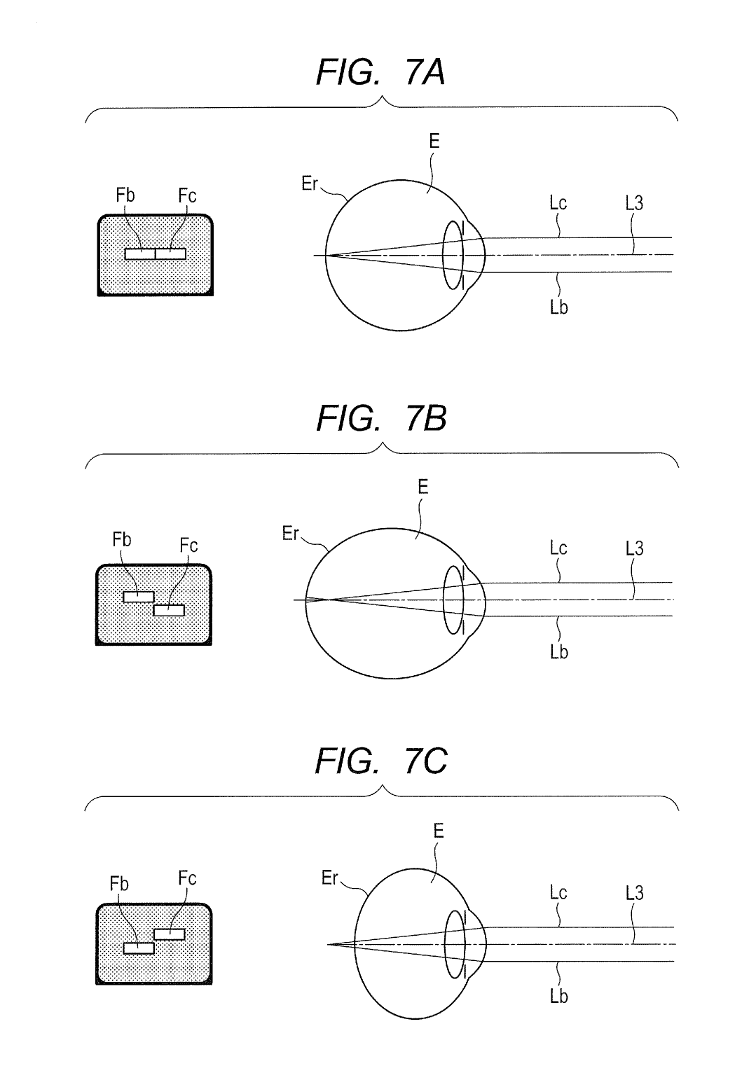

To easily focus on the fundus of the eye to be inspected, the focus index split on the pupil of the eye to be inspected is projected to the fundus of the eye to be inspected. The return light therefrom is guided to the image pickup element 14 via the optical axis L3. An appearance of the image pickup element 14 is displayed on the monitor 15 via the image processing portion 17. FIGS. 7A to 7C are schematic diagrams illustrating the relation between the appearance of the focus index image displayed on the monitor 15 and a state in which focus index light beams Lb and Lc reach the fundus Er of the eye to be inspected.

FIG. 7A illustrates a case in which the fundus Er of the eye E to be inspected is optically in the conjugate positional relation with the focus index of the focus index projection portion 22. Because the fundus Er is optically in the conjugate positional relation with the focus index, the focus index light beams Lb and Lc split into two become index images Fb and Fc to be aligned in a line on the fundus Er.

FIG. 7B illustrates a case in which the eye E to be inspected has a higher degree of myopia than in FIG. 7A. At this juncture, because the fundus Er and the focus index are not optically conjugated, the focus index light beams Lb and Lc split into two become focus index images Fb and Fc on the fundus Er. The focus index image Fb is shifted upwardly and the locus index image Fc is shifted downwardly.

FIG. 7C illustrates a case in which the eye E to be inspected has a higher degree of hyperopia than in FIG. 7A. Because the fundus Er and the focus index are not optically conjugated, the focus index light beams Lb and Lc split into two become focus index images Fb and Fc on the fundus Er. The focus index image Fb is shifted downwardly and the focus index image Fc is shifted upwardly.

To align the focus index images Fb and Fc in a line, that is, to make the fundus Er be optically conjugated with the focus index, the respective gravity centers of the focus index images Fb and Fc are acquired and the difference thereof is calculated. Further, the reason why the system control portion 18 dims the observation light source 1 in the first control (S204) is described.

The timing for executing the first control (S204) is after executing the operation of retracting the anterior ocular segment observation lens 23 from above the optical axis L3 (S203). Because this is the timing of switching from the anterior ocular observation mode to the fundus observation and photographing mode, a sense of incompatibility in appearance is hard to be felt for the examiner. Further, because the image displayed on the monitor 15 after the first control (S204) is an image put into focus, the examiner can smoothly move into the fundus observation and photographing mode.

Further, even the focus index in which the positional difference of the focus index is large and thus blurred and low in luminance degree can sufficiently recognize the difference in luminance degree between the focus index and the observation light by keeping the observation light 1 dimmed, and therefore, there is an advantage that a probability of accurately succeeding in detecting the positional difference of the focus index is increased.

Subsequently, the examiner refers to a fundus alignment index projected to a cornea Ep of the eye to be inspected via the fiber 25 while observing the eye E to be inspected displayed on the monitor 15, and executes again the alignment between the eye E to be inspected and the fundus camera (S205).

In a second control (S207), the focus index image of the focus index projection portion 22 photographed by the image pickup element 14 is acquired (S214), and the system control portion 18 calculates the positional difference of the focus index image (S215). Based on the calculation result, the system control portion 18 controls the focus lens drive portion 19 and the focus index drive portion 20 (S216). After controlling both drive portions 19 and 20, the system, control portion 18 stores the current focus position (S217), thereby completing the second control.

The first control and the second control are different in the presence or absence of the dimming of the fundus observation light. The second control is executed before photographing. In this case, the ophthalmologic apparatus which refers to a photometric value and determines a photographing light intensity is unable to acquire a correct photometric value when the control is executed under the dimming. For the above-mentioned reason, the second control does not dim the fundus observation light.

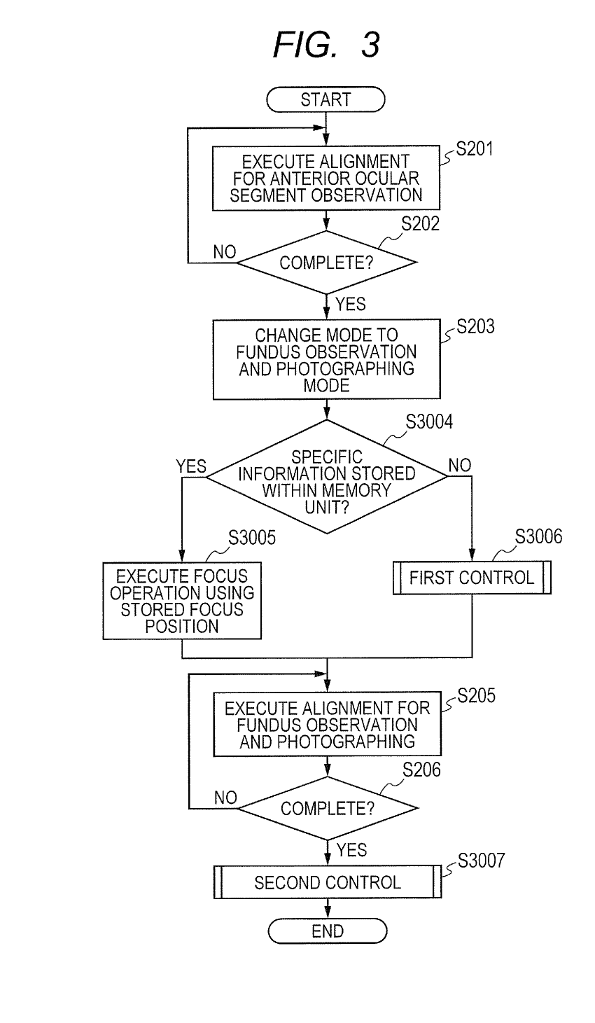

Next, with reference to FIG. 3 while comparing to the flow illustrated in FIG. 2A, a fundus examination operation in the case of storing the specific information in the memory unit and not storing is described.

In FIG. 3, Operations on S201 to S203 show the same details as details shown by S201 to S203 of FIG. 2A, and operations on S205 and S206 in FIG. 3 show the same details as details shown, by S205 and S206 of FIG. 2A. In S3004, when the specific information on the eye to be inspected is stored in the memory unit, the system control portion 18 controls the focus lens drive portion 19 and the focus index drive portion 20 to the stored focus position (S3005). When not stored, the first control is executed (S3006).

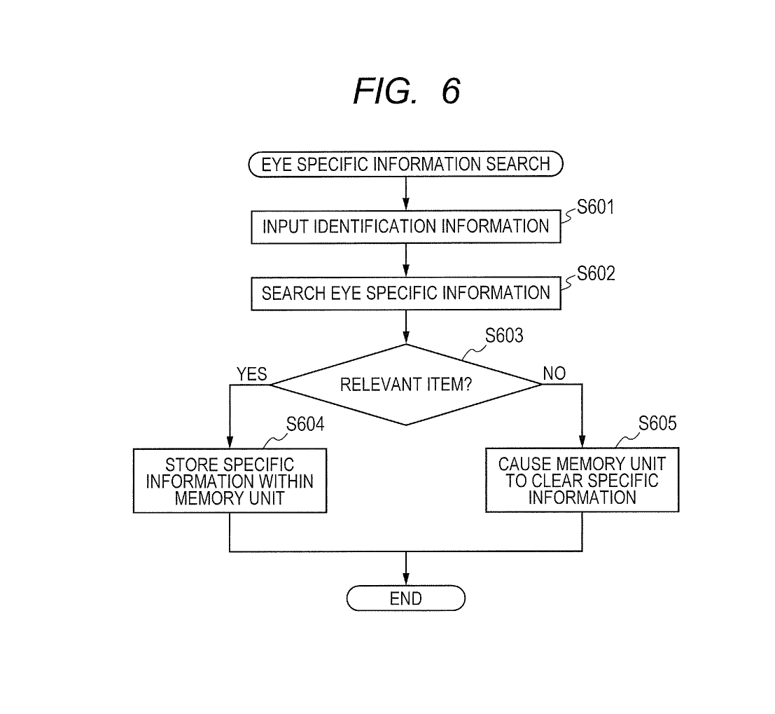

Note that, the processing for storing the specific information in the memory unit has a method to be executed within the first control (S3006), a method to be executed, within the second control (S3007), and a method to be described below with reference to FIG. 6. The first control and the second control described herein are basically the same as the first control and the second control shown in FIGS. 2B and 2C, respectively.

Next, the operation in the case of photographing the eye to be inspected of the same subject again is described.

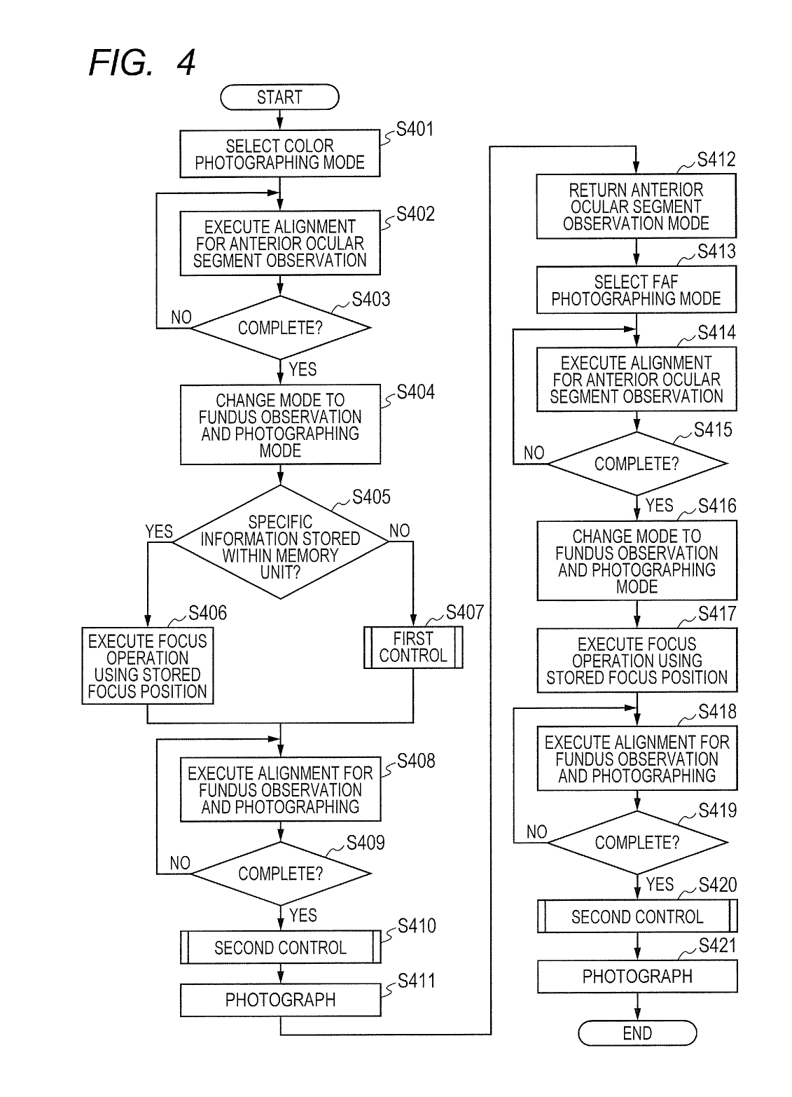

FIG. 4 is a diagram illustrating a photographing flow in the case of multiple photographing of the same eye of the same subject. For example, color photographing is executed the first time, and the FAF photographing using autofluorescence of the fundus of the eye to be inspected is executed the second time.

The examiner presses a photographing selection switch (not shown) to select a color photographing mode (S401). The examiner executes the alignment between the anterior ocular segment of the eye E to be inspected and the fundus camera with respect to a horizontal direction (X direction), a vertical direction (Y direction), and a front-back direction (Z direction), while observing the anterior ocular segment of the eye E to be inspected displayed on the monitor 15 (S402). When the alignment between the anterior ocular segment of the eye E to be inspected and the fundus camera is completed (S403), the examiner executes the operation of retracting the anterior ocular segment observation lens 23 from above the optical axis L3 (S404).

At S405, when the specific information on the eye to be inspected is stored in the memory unit 26, the system control portion 18 controls the focus lens drive portion 19 and the focus index drive portion 20 to the stored focus position (S406). When not stored, the first control is executed (S407).

The examiner refers to a fundus alignment index projected to the cornea Ep of the eye to be inspected via the fiber 25 while observing the eye E to be inspected displayed on the monitor 15, and executes the alignment between the eye E to be inspected and the fundus camera (S408). In the second control (S410), the focus index image of the focus index projection portion 22 photographed by the image pickup element 14 is acquired (S214), and the system control portion 18 calculates the positional difference of the index image (S215). Based on the calculation result, the system control portion 18 controls the focus lens drive portion 19 and the focus index drive portion 20 (S216). After controlling both drive portions 19 and 20, the system control portion 18 stores the current focus position (S217), thereby completing the second control. After the second control, photographing is executed (S411). After photographing, this fundus camera returns to the anterior ocular segment observation mode (S412). At this juncture, the focus lens 12 is driven to the position of 0 Diopter.

Subsequently, the examiner presses a photographing selection switch (not shown) to select an FAF photographing mode (S413). The examiner executes the alignment between the anterior ocular segment or the eye E to be inspected and the fundus camera with respect to a horizontal direction (X direction), a vertical direction (Y direction), and a front-back direction (Z direction), while observing the anterior ocular segment of the eye E to be inspected displayed on the monitor 15 (S414). When the alignment between the anterior ocular segment of the eye E to be inspected and the fundus camera is completed (S415), the examiner executes the operation of retracting the anterior ocular segment observation lens 23 from above the optical axis L3 (S416). At S417, the focus lens 12 is driven to a focus position stored within the second control of S410. The examiner refers to a fundus alignment index projected to the cornea Ep of the eye to be inspected via the fiber 25 while observing the eye E to be inspected displayed on the monitor 15, and executes the alignment between the eye E to be inspected and the fundus camera (S418). In the second control (S420), the focus index image of the focus index projection portion 22 photographed by the image pickup element 14 is acquired (S214), and the system control portion 18 calculates the positional difference of the index image (S215). Based on the calculation result, the system control portion 18 controls the focus lens drive portion 19 and the focus index drive portion 20 (S216). After controlling both drive portions 19 and 20, the system control portion 18 stores the current focus position (S217), thereby completing the second control. After the second control, photographing is executed (S421), thereby completing this photographing flow.

In other words, the system control portion 18 functions as a focus control unit in the present invention, and changes light intensity into a first light intensity by a module region that functions as the light intensity control unit. Further, the first control which controls the focus lens drive portion 19 to drive the focus lens 12 based on the index image acquired in a state where the light intensity is set to the first light intensity and the second control which sets the illumination light intensity to a second light intensity higher than the first light intensity and drives the focus lens 12 based on the index image acquired in a state where the light intensity is set to the second light intensity are executed. Further, the system control portion 18 also includes a module region that functions as a memory control unit which stores the specific information in the memory unit 26 during the first and second controls. The system control portion 18 executes the second control after driving the focus lens 12 to the position stored in the memory unit 26 when the specific information is stored again in the memory unit 26 after storing further the specific information in the memory unit 26 as the memory control unit.

Note that, after photographing at S411, the fundus camera may not be returned to the anterior ocular segment observation mode, but remain as it is at the fundus observation and photographing mode. In this case, the photographing flow is started from S418, and the specific information on the eye to be inspected may be updated within the second control of S416.

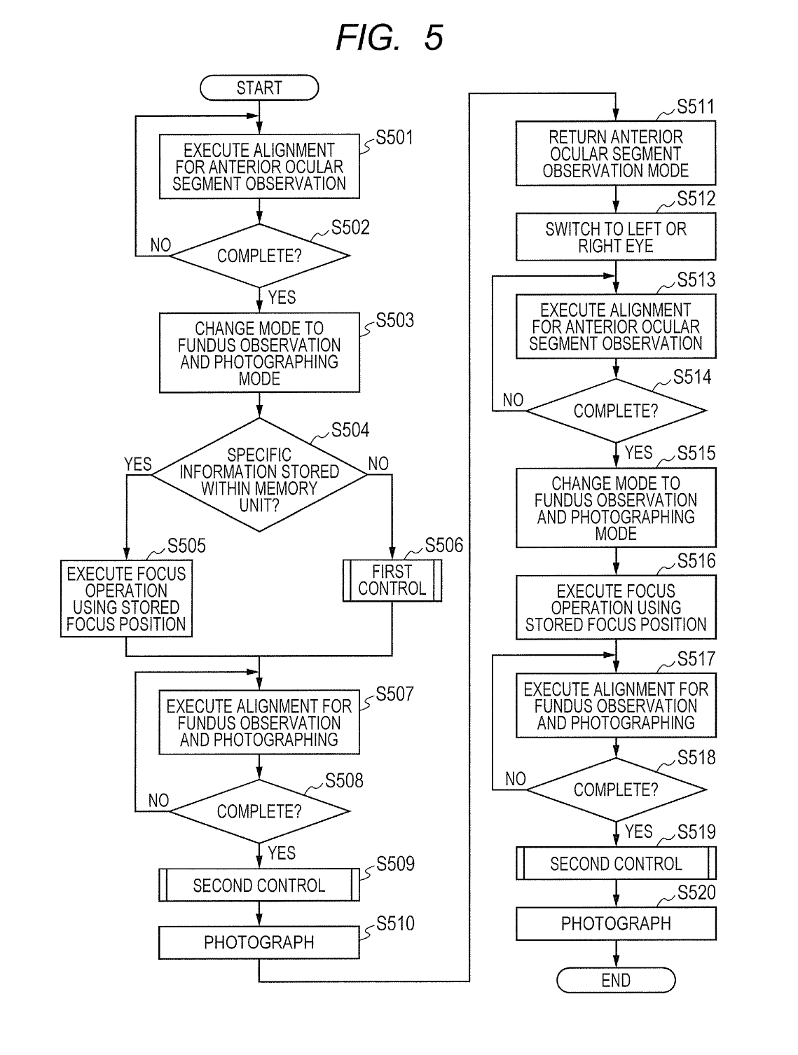

FIG. 5 is a diagram illustrating the photographing flow when left/right eye switching is executed after photographing the fundus of the same subject.

Operations on S501 to S510 are as described previously using S402 to S411 of FIG. 4. After the operation of left/right eye switching by the examiner is executed so as to photograph an eye opposite to the eye previously photographed (S512), the examiner executes the alignment between the anterior ocular segment of the eye E to be inspected and the fundus camera with respect to a horizontal direction (X direction), a vertical direction (Y direction), and a front-back direction (Z direction), while observing the anterior ocular segment of the eye E to be inspected displayed on the monitor 15 (S513). When the alignment between the anterior ocular segment of the eye E to be inspected and the fundus camera is completed (S514), the examiner executes the operation of retracting the anterior ocular segment observation lens 23 from above the optical axis L3 (S515). Immediately after the processing of S515, the fundus camera drives the focus lens 12 and the focus index projection portion 22 to the focus position which stores the specific information on the eye to be inspected (S516). The examiner refers to a fundus alignment index projected to the cornea Ep of the eye to be inspected via the fiber 25 while observing the eye E to be inspected displayed on the monitor 15, and executes the alignment between the eye E to be inspected and the fundus camera (S517). In the second control (S519), the focus index image of the focus index projection portion 22 photographed by the image pickup element 14 is acquired (S214), and the system control portion 18 calculates the positional difference of the index image (S215). Eased on the calculation result, the system control portion 18 controls the focus lens drive portion 19 and the focus index drive portion 20 (S216). After controlling both drive portions 19 and 20, the system control portion 18 stores the current focus position (S217), thereby completing the second control. After the second control, photographing is executed (S520), thereby completing this photographing flow.

FIG. 6 is a diagram illustrating the operation at the time of searching for the specific information on the eye to be inspected by a search unit.

Identification information on the subject is input by an input unit such as a key board and a mouse (not shown) (S601), and the specific information on the eye to be inspected is searched for (S602). As a result of searching (S603), when the relevant item is available, the specific information on the eye to be inspected is stored in the memory unit 26 (S604). When there is no relevant item available, the specific information on the eye to be inspected stored in the memory unit 26 is cleared (S605).

### Other Embodiment

Further, the present invention may also be realized by executing the following process. Specifically, software (program) for realizing the function of the embodiment described above is supplied to a system or an apparatus via a network or an arbitrary type of storage medium, and a computer (CPU or MPU) of the system or the apparatus reads and executes the program.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-247026, filed Nov. 9, 2012, which is hereby incorporated by reference herein in its entirety.

What is claimed is:

1. An ophthalmologic apparatus, comprising:
a memory unit for storing specific information on an eye to be inspected;
an illumination unit for illuminating the eye to be inspected;
a projection unit for projecting an index to the eye to be inspected;
an index image acquire unit for acquiring an index image based on return light from the eye to be inspected;
a drive unit for driving a focus unit based on the index image;
a light intensity control unit for controlling light intensity that illuminates the eye to be inspected from the illumination unit;
a focus control unit for executing a first control for controlling the light intensity control unit to change the light intensity into a first light intensity and controlling the drive unit to drive the focus unit based on the index image acquired in a state of changing the light intensity into the first light intensity and a second control for controlling the light intensity control unit to change the light intensity into a second light intensity higher than the first light intensity and controlling the drive unit to drive the focus unit based on the index image acquired in a state of changing the light intensity into the second light intensity; and
a memory control unit for controlling the memory unit to store the specific information during the first control and the second control;
wherein the focus control unit executes the second control after the focus unit is driven to a position stored in the memory unit in a case where the memory control unit controls the memory unit to store the specific information and thereafter controls the memory unit to store the specific information again.

2. An ophthalmologic apparatus according to claim 1, further comprising;
an input unit for inputting identification information on the eye to be inspected; and
a search unit for searching for the specific information associated with the input identification information,
wherein the memory unit stores the specific information in association with the identification, information on the eye to be inspected, and
wherein the drive unit drives the focus unit to a position based on the retrieved specific information.

3. An ophthalmologic apparatus according to claim 2, wherein the specific information comprises a position of the focus lens when stored in the memory unit one of during the first control, during the second control, and when there is a relevant item made available by the search unit.

4. An ophthalmologic examination method, comprising:
a first control step of changing light intensity for illuminating an eye to be inspected into a first light intensity and driving a focus unit based on an index image acquired by projecting an index to the eye to be inspected in a state of changing the light intensity into the first light intensity;
a second control step of changing the light intensity into a second light intensity higher than the first light intensity and driving the focus unit based on the index image acquired in a state of changing the light intensity into the second light intensity; and
a memory step of storing specific information on the eye to be inspected during the first control step and the second control step,
wherein the second control step is executed after driving the focus unit to a position stored in the memory step in a case where the specific information is stored again after the specific information is stored in the memory step.

5. An ophthalmologic examination method according to claim 4, further comprising:
an input step of inputting identification information on the eye to be inspected; and
a search step of searching for the specific information associated with the input identification information,
wherein the memory step comprises storing the specific information in association with the identification information on the eye to be inspected, and
wherein the focus unit is driven to a position based on the retrieved specific information.

6. An ophthalmologic examination method according to claim 5, wherein the specific information comprises a position of the focus lens when stored in the memory step one of during the first control step, during the second control step, and when there is a relevant item made available in the search step.

7. A program for controlling a computer to execute the steps of the ophthalmologic examination method according to claim A.
